(19) United States
(12) Patent Application Publication
(10) Pub. No.: US 2007/0055329 A1
(43) Pub. Date: Mar. 8, 2007

(54) Thermal sleeve and wrap

(75) Inventors: George T. Hicks, Walnut Cove, NC (US); Larry G. Reid JR., Germanton, NC (US)

Correspondence Address: WHITE, REDWAY & BROWN LLP, 1217 KING STREET, ALEXANDRIA, VA, 22314, US

(73) Assignee: Carolon Company, Rural Hall, NC (US)

(21) Appl. No.: 11/491,491

(22) Filed: Jul. 24, 2006

Related U.S. Application Data
(60) Provisional application No. 60/701,513, filed on Jul. 22, 2005.

(51) Int. Cl. A61F 7/00

(52) U.S. Cl. 607/112; 607/114

(57) ABSTRACT

An article for applying hot or cold therapy to an individual in need of such therapy which includes a sleeve containing a thermal pack encased within a moisture absorbing fabric and a temperature control fabric adjacent the thermal pack.

This application claims priority under 35 U.S.C. § 119 to U.S. provisional application No. 60/701,503 filed Jul. 22, 2005, the entire content of which is incorporated herein by reference.

### BACKGROUND OF THE INVENTION

The present invention relates to a multilayered fabric designed to regulate and control the transfer of heat and moisture on the external surface of an individual when used in conjunction with hot and cold therapy packs, to such hot and cold packs, and to a method for treating an individual with hot or cold therapy.

Hot and cold packs have been used to treat a variety of conditions such as, inter alia, muscle and tendon injuries, chronic pain, and joint inflammation. The pack itself is usually held in place on a body surface by means of a wrap of fabric. Although known hot and cold packs are very similar and usually comprise gel packs contained in a bag of nylon, polyester, vinyl, or other woven on non-woven polymeric material, the prior art fabrics used between the pack and body fail to enhance the effectiveness of the pack and/or do not provide adequate protection for the user. For example, excessive cold can cause frostbite without the proper insulation. Inadequate moisture control of a hot pack can result in inadequate heat transfer from the pack to the body surface.

### SUMMARY OF THE INVENTION

It is according an aspect of the invention to provide a fabric composition which affords increased control of heat transfer from a hot or cold pack incorporating such fabric.

It is another aspect of the invention to provide hot and cold packs incorporating such fabric compositions.

It is yet another aspect of the invention to provide a method for treating an individual using the above hot and cold packs.

These aspects and others set forth below, are achieved by a sleeve which comprises a thermal pack encased within a moisture absorbing fabric. A temperature control fabric is positioned adjacent the thermal pack along one side thereof.

### BRIEF DESCRIPTION OF THE DRAWINGS

FIG. 1 is a cross-sectional view of one embodiment of the sleeve of thg invention incorporating a thermal pack; and

FIG. 2 is a cross-sectional view of one embodiment of the wrap of the invention including a thermal pack and a temperature control fabric.

### DETAILED DESCRIPTION OF THE INVENTION

The choice and configuration of the fabric can have a significant impact on the effectiveness of the pack in use. Important parameters of the fabric include insulation qualities and heat distribution, moisture control and comfort.

With these parameters in mind, the inventors have found that a combination of at least two layers of fabric, each having a different function, can simultaneously provide moisture control and heat distribution over a wide temperature range. This result was unexpected, since it was heretofore believed. that moisture control and heat distribution were mutually exclusive in a single wrap.

In a preferred embodiment, suitable yarn for temperature control has thermal properties which vary as a function of temperature. Standardized measurement of thermal properties of yarn under static and dynamic temperature conditions is provided in accordance with Test Method for Steady State and Dynamic Thermal Performance in Textile Materials (ASTM D7024). In a highly preferred embodiment, the temperature control yarn incorporates one or more phase change materials which act as temperature buffers. Such materials undergo a phase change as the surrounding temperature varies, absorbing heat during a temperature rise, and releasing heat when temperature falls. The melting point of the phase change materials, that is, the temperature at which the phase change occurs, can be varied depending on the particular application. A source for suitable fabrics incorporating phase change materials is Outlast Technologies of Boulder, Colo.

In addition to the selection of particular phase change materials, the construction of the temperature control yarn can be varied by the use of specific denier and loop size in the terry.

Moisture control can be accomplished by the use of a wicking yarn effectively moving the moisture to enhance effectiveness and safety. Various moisture wicking yarns are available, one of which is Sorbtek manufactured by Unifi.

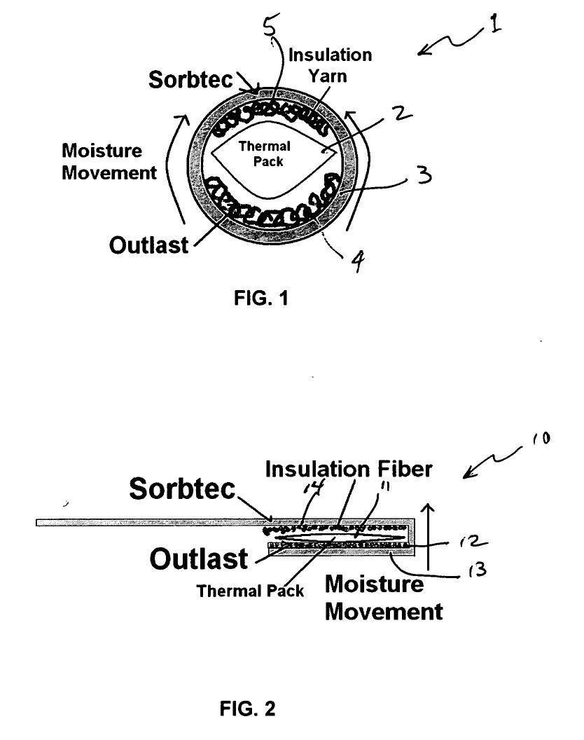

FIG. 1 illustrates one embodiment of the invention comprising a sleeve 1 in which a thermal pack 2 is encased within a moisture absorbing fabric 3. A temperature control fabric 4 is positioned adjacent the thermal pack along one side thereof. In an alternative embodiment, an insulation fabric can be positioned adjacent the pack on a side opposite the temperature control fabric 4.

The sleeve 1 is designed to be placed directly over the affected area, for example, a knee. The sleeve is oriented with the temperature control fabric positioned between the affected area and the thermal pack 2. The optional insulation fabric reduces heat loss from the thermal pack from the side opposite to that applied to the affected area.

FIG. 2 illustrates another embodiment of the invention comprising a wrap 10. The wrap 10 includes a thermal pack 11 and a temperature control fabric 12. The pack 11 and fabric 12 are contained in a sleeve 13 comprising a moisture absorbing fabric 13. In an alternative embodiment (not shown), a sleeve of suitable fabric can be knitted on a circular knitting machine and a moisture absorbing fabric is plated to the outside of the sleeve and a temperature control fabric is constructed with a terry knit to the inside of the sleeve. The moisture absorbing material can also knitted around the entire circumference with the terry covering half the circumference. Additional yarns such as various well know acrylics may be added to manipulate the fabric weight.

The wrap can be constructed using a flat knit machine or weaving. The two layers of the moisture absorbing fabric and temperature control fabric can be woven using a Jacquard machine or the fabrics can be woven separately and then sewn together.

A thermocromatic ink can be printed on the fabric to indicate to the user the temperature of the pack when it is heated such as in a microwave or cooled such as a freezer. This would indicate that the pack has reached the proper temperature, that it is too hot or that it is sufficiently cold.

What is claimed is:

1. A sleeve for external application of hot or cold therapy, comprising:
a thermal pack encased within a moisture absorbing fabric; and
a temperature control fabric positioned adjacent the thermal pack along one side thereof.

2. A sleeve as claimed in claim 1, further comprising an insulating yain positioned adjacent said thermal pack on a side opposite that of said temperature control fabric.

3. A sleeve as claimed in claim 1, wherein said sleeve provides hot therapy.

4. A sleeve as claimed in claim 1, wherein said sleeve provides cold therapy.

5. A wrap for external application of hot or cold therapy, comprising:
a sleeve comprising a thermal pack encased within a moisture absorbing fabric, and a temperature control fabric positioned adjacent the thermal pack along one side thereof; and
a fabric portion extended from said sleeve and suitable for wrapping around and external surface.

6. A sleeve as claimed in claim 5, wherein said sleeve provides hot therapy.

7. A sleeve as claimed in claim 5, wherein said sleeve provides cold therapy.

8. A method for treating an individual with hot or cold therapy, comprising the steps of:
providing a sleeve comprising a thermal pack encased within a moisture absorbing fabric, and a temperature control fabric positioned adjacent the thermal pack along one side thereof; and
positioning said sleeve adjacent to or directly on the exterior surface of the individual; and
transferring heat or cold to the external surface of said individual via said sleeve.
